(19) United States
(12) Patent Application Publication
(10) Pub. No.: US 2021/0246466 A1
(43) Pub. Date: Aug. 12, 2021

(54) REGULATABLE GENE EDITING COMPOSITIONS AND METHODS

(71) Applicant: The Trustees of the University of Pennsylvania, Philadelphia, PA (US)

(72) Inventors: James M. Wilson, Philadelphia, PA (US); Lili Wang, Phoenixville, PA (US)

(21) Appl. No.: 16/610,846

(22) PCT Filed: May 3, 2018
(86) PCT No.: PCT/US2018/030868

§ 371 Date: Nov. 4, 2019

Related U.S. Application Data
(60) Provisional application No. 62/501,338, filed on May 4, 2017.

(51) Int. Cl. C12N 15/86; A61K 35/76; C12N 9/22; C07K 14/47

(52) U.S. Cl. CPC C12N 15/86; A61K 35/76; C12N 9/22; C07K 14/4702; C12N 2310/20; C12N 2830/008; C12N 2840/203; C07K 2319/81; C12N 2800/80; C12N 2750/14143

(57) ABSTRACT

Provided herein is a regulatable gene editing system. The system includes at least one regulatable promoter which controls expression of a gene editing nuclease. The system may optionally contain more than one regulatable promoter, e.g., one promoter for the guide RNA where the system is a CRISPR system and another promoter for a selected gene. The system involves delivering to a subject: (a) at least one nucleic acid encoding one or more DNA binding domains, (b) a nucleic acid sequence comprising a donor gene for insertion into a selected gene locus, (c) at least one nucleic acid sequence comprising a coding sequence of an activation domain for the regulatable promoter, and (d) at least one coding sequence encoding a nuclease; wherein expression of the nuclease is under the control of at least one regulatable promoter, and the promoter is activated and/or regulated by a pharmaceutical agent. Also provided are methods for treating disorders associated with specific genetic abnormalities by correcting or replacing the gene mutation or defect.

### BACKGROUND OF THE INVENTION

Early intervention and therapy is crucial in many inherited diseases. Various types of therapies have been described in the literature. One technique which has been described as having potential in correction of diseases associated with a genetic mutation or a specific phenotype is genome editing. Genome editing techniques have been described in the literature, including the use of transcription activator-like effector (TALE) nucleases (TALENs), zinc finger nucleases (ZFNs), engineered meganucleases, and the clustered, regularly interspaced short palindromic repeats (CRISPR) systems.

Meganucleases have been used extensively for genome editing in a variety of different cell types and organisms. Meganucleases are engineered versions of naturally occurring restriction enzymes that typically have extended DNA recognition sequences (e.g., 14-40 bp). ZFNs and TALENs are artificial fusion proteins composed of an engineered DNA binding domain fused to a nonspecific nuclease domain from the FokI restriction enzyme. Zinc finger and TALE repeat domains with customized specificities can be joined together into arrays that bind to extended DNA sequences. CRISPR-Cas was derived from an adaptive immune response defense mechanism used by archaea and bacteria for the degradation of foreign genetic material [Van der Oost, J., et al. 2014. Nat. Rev. Microbiol. 7: 479-492; Hsu, P., et al. 2014. Development and applications of CRISPR-Cas9 for genome editing. Cell 157: 1262-1278]. This mechanism can be repurposed for other functions, including genomic engineering for mammalian systems, such as gene knockout (KO) [Cong, L., et al. 2013. Multiplex genome engineering using CRISPR/Cas systems. Science 339: 819-823; Mali, P., et al. 2013. RNA-guided human genome engineering via Cas9. Science 339: 823-826; Ran, F. A., et al. 2013. Genome engineering using the CRISPR-Cas9 system. Nat. Protoc. 8: 2281-2308; Shalem, O., et al. 2014. Genome-scale CRISPR-Cas9 knockout screening in human cells. Science 343: 84-87]. The CRISPR Type II system is currently the most commonly used RNA-guided endonuclease technology for genome engineering. There are two distinct components to this system: (1) a guide RNA and (2) an endonuclease, such as the CRISPR associated (Cas) nuclease, Cas9. The guide RNA (gRNA) is a combination of the endogenous bacterial crRNA (CRISPR RNA) and tracrRNA (transactivating crRNA) into a single chimeric gRNA transcript. The gRNA combines the targeting specificity of crRNA with the scaffolding properties of tracrRNA into a single transcript. When the gRNA and the Cas9 are expressed in the cell, the genomic target sequence can be modified or permanently disrupted.

A number of concerns have been raised regarding genome editing techniques, including safety concerns regarding unexpected toxicity in a host. A need for improved genome editing systems remains in the art.

### SUMMARY OF THE INVENTION

Compositions and methods that allow for temporal control of the activity of the editing nucleases are provided. The system can be delivered using viral and non-viral delivery vehicles. CRISPR-like nucleases, meganucleases, zinc finger nucleases, and other types of nucleases are expressed under control of a regulatable promoter. The system may include additional elements (e.g., gRNA) expressed under the control of regulatable promoters. In certain embodiments, a gRNA is expressed under the control of a promoter specific for the target tissue (e.g., a liver-specific promoter).

In one aspect, a regulatable gene editing system is provided for treating disorders. The system comprises: (a) at least one nucleic acid sequence encoding one or more DNA binding domains; (b) at least one nucleic acid sequence comprising a coding sequence of an activation domain for the regulatable promoter; (d) at least one coding sequence encoding a nuclease; and (d) optionally, a nucleic acid sequence comprising a donor gene for insertion into a selected gene locus; wherein expression of the nuclease is under the control of at least one regulatable promoter which is activated and/or regulated by a pharmaceutical agent. In certain embodiments, the gene editing system comprises: (a) one or more nucleic acid molecules comprising a gene editing nuclease gene under control of a regulatable promoter which directs its expression in a target cell (e.g., a hepatocyte) and further comprising a targeted gene which has one or more mutations resulting in a disease or disorder (e.g., a liver metabolic disorder); (b) one or more nucleic acid sequences comprising specific DNA binding domains and a donor template, wherein the DNA binding domains specifically bind to a selected site in the targeted gene and is 5′ to a motif which is specifically recognized by the nuclease; and (c) optionally one or more coding sequences for a therapeutic gene.

In certain embodiments, the system uses a meganuclease under the control of a rapamycin-regulatable promoter. In certain embodiments, the methods and compositions use one or more recombinant adeno-associated virus (AAV) vectors.

In one aspect, a dual vector system for treating disorders is provided, wherein the system comprises: (a) a gene editing vector comprising a Cas9 gene under the control of a regulatable promoter which directs its expression in a target cell (e.g., a hepatocyte) comprising a targeted gene which has one or more mutations resulting in a disease or disorder (e.g., a liver metabolic disorder); and (b) a targeting vector comprising one or more of sgRNAs and a donor template, wherein the sgRNA comprises at least 20 nucleotides which specifically bind to a selected site in the targeted gene and is 5′ to a protospacer-adjacent motif (PAM) which is specifically recognized by the Cas9, and wherein the donor template comprises nucleic acid sequences which replace at least one of the mutations in the targeted gene; wherein the ratio of gene editing vector (a) to the vector containing template (b) is such that (b) is in excess of (a). In certain embodiments, the disorder is a metabolic disorder. In another embodiment, the disorder is a liver metabolic disorder. In certain embodiments, the vectors used in this system are AAV vectors. In one example, both the gene editing AAV vector and the targeting AAV vector have the same capsid. Optionally, the sgRNA may also be under the control of a regulatable promoter, such as described herein.

Still other aspects and advantages of the invention will be readily apparent from the following detailed description of the invention.

### BRIEF DESCRIPTION OF THE DRAWINGS

FIGS. 1A and 1B illustrate a two-vector system suitable for an AAV vector and designed for liver-targeted therapy (liver-specific promoter selected). FIG. 1A is a schematic for a transcription factor vector which contains, from 5′ to 3′: a 5′-ITR, a liver-specific promoter operably linked to an FRB-p65 activation domain fusion protein, a linker (IRES), a DNA binding domain fusion protein, and a human growth hormone 3′ UTR, followed by a 3′-ITR. ZFHD refers to a DNA binding domain composed of a zinc finger pair and homeodomain (ZFHD1). FIG. 1B is a schematic for a target gene vector which contains, from 5′ to 3′: a 5′-ITR, 12 zinc finger HD1 sites, a minimal IL2 promoter operably linked to a meganuclease coding sequence, a woodchuck post-regulatory element (WPRE), a bovine growth hormone polyA (bGH pA), and a 3′-ITR.

FIG. 2 illustrates a one-vector system designed for liver-targeted therapy. This system includes, from 5′ to 3′: an ITR, a liver-specific promoter which directs control of an activation domain fusion protein, a linker, a DNA binding domain fusion, a human GH poly A, eight zinc finger binding sites, a minimum IL2 promoter operably linked to a meganuclease coding sequence, a polyA, and an ITR. In the interest of space, this vector utilizes one fewer FKBP and four fewer zinc finger sites as compared to the two-vector system. However, the number of FKBP and ZFHD1 may be further altered.

FIGS. 3A and 3B illustrate a two-vector system suitable for liver-targeted therapy in which the gene editing nuclease is Cas9. FIG. 3A is a schematic for a transcription factor vector which contains, from 5′ to 3′: a 5′-ITR, a liver-specific promoter operably linked to an FRB-p65 activation domain fusion protein, a linker, a DNA binding domain fusion protein, and a human growth hormone 3′ UTR, followed by a 3′-ITR. FIG. 3B is a schematic for a target gene vector which contains, from 5′ to 3′: a 5′-ITR, 12 ZFHD1 binding sites, a minimal IL2 promoter operably linked to a Cas9 coding sequence, a polyA, and a 3′-ITR.

### DETAILED DESCRIPTION OF THE INVENTION

A system is provided herein in which a gene editing nuclease is expressed in vivo under the control of a regulatable promoter. This improves control and safety, permitting temporal control (i.e., control of the timing of induction). This may be an important feature which adapts to the kinetics of the delivery method used for the genome editing system. For example, in an AAV-based system, it may be desirable to defer induction of the nuclease until about 3 days to about 14 days post-dosing, although shorter or longer times may be used. Further, by controlling the dose of the inducing agent, the kinetics of genome editing may be controlled as well. Thus, relatively low doses of inducing agent may be delivered daily, or there may be breaks of one, two, three, seven, 14 or more days between doses of inducing agent. However, for other delivery methods (e.g., physical methods) induction may be essentially simultaneous, or within about 24 hours of dosing the patient. Other suitable timelines for providing the inducing agent may be selected by one of skill in the art.

Thus, provided herein is a method of treatment using a regulatable gene editing system. The system includes at least one regulatable promoter which controls expression of a gene editing nuclease. The system may optionally include more than one regulatable promoter, e.g., one for the gRNA where the system is a CRISPR system and another for the selected nuclease. In certain embodiments, the system includes delivering to a subject: (a) one or more DNA binding domains, (b) a nucleic acid sequence comprising a donor gene for insertion into a selected gene locus; (c) at least one nucleic acid sequence comprising a coding sequence of an activation domain for the regulatable promoter; (d) at least one coding sequence encoding a nuclease; wherein expression of the nuclease is under the control of at least one regulatable promoter, wherein the promoter is activated and/or regulated by pharmaceutical agent. Also provided are methods for treating disorders associated with specific genetic abnormalities by correcting or replacing the gene mutation or defect.

As used herein, a gene editing nuclease may include, e.g., a meganuclease (recombinant, native, or engineered), a zinc finger nuclease, a TALEN, or a CRISPR associated nuclease.

As used herein, the zinc finger nuclease (ZFN) cleaves a target genomic region of interest, wherein the ZFN comprises one or more engineered zinc-finger binding domains and a nuclease cleavage domain or cleavage half-domain. Cleavage domains and cleavage half-domains can be obtained, for example, from various restriction endonucleases and/or homing endonucleases. In certain embodiments, the cleavage half-domains are derived from a Type IIS restriction endonuclease (e.g., Fok I). In certain embodiments, the zinc finger domain recognizes a target site in a disease associated gene (See, e.g., U.S. Pat. No. 9,315,825, which is incorporated herein by reference).

As used herein, a transcription activator-like effector nuclease (TALEN) cleaves a target genomic region of interest, wherein the TALEN comprises one or more engineered TALE DNA binding domains and a nuclease cleavage domain or cleavage half-domain. Cleavage domains and cleavage half-domains can be obtained, for example, from various restriction endonucleases and/or homing endonucleases. In certain embodiments, the cleavage half-domains are derived from a Type IIS restriction endonuclease (e.g., Fok I). In certain embodiments, the TALE DNA binding domain recognizes a target site in a highly expressed, disease associated gene.

In certain embodiments, a CRISPR/Cas system binds to target site in a region of interest (e.g., a highly expressed gene, a disease associated gene, or a safe harbor gene) in a genome, wherein the CRISPR/Cas system comprises a CRIPSR/Cas nuclease and an engineered crRNA/tracrRNA (or single guide RNA). In certain embodiments, the CRISPR/Cas system recognizes a target site in a highly expressed, disease associated gene. See, e.g., WO 2016/176191, which is incorporated herein by reference. In certain embodiments, the Cas9 enzyme is used in the CRISPR system. In other embodiments, the CpfI enzyme may be used.

As used herein, a meganuclease includes homing endonucleases, which can be divided into five families based on the following sequence and structural motifs: LAGLIDADG, GIY-YIG, HNH, His-Cys box and PD-(D/E)XK. See, e.g., U.S. Pat. No. 8,338,157, which is incorporated by reference herein, describing engineered meganucleases of the “LIG-34 meganucleases”. See also, U.S. Pat. Nos. 9,434,931, 9,340,077, 8,445,251, and 8,304,222 describing rationally designed LAGLIDADG meganucleases, which are incorporated herein by reference.

Both physical and non-physical methods and delivery vectors may be used for the delivery of a nuclease-based genome editing system. In physical methods, such as microinjection, electroporation, ballistic delivery, and laser, physical energy is used for cell entry. In non-physical systems, vectors, including both viral vectors and non-viral vectors, can encapsulate the plasmid or mRNA of these programmable nucleases or nuclease proteins, and carry them into target tissues or cells. Vectors used for gene-based systemic delivery may include non-viral vectors, such as lipid nanoparticles (LNPs), liposomes, polymers, conjugates, and cell-derived membrane vesicles (CMVs), or viral delivery systems, including viral vectors, such as lentivirus vectors (LVs), adenovirus vectors (AdVs), adeno-associated virus vectors (AAVs), and herpes simplex-1 virus vectors (HSV-1s). Optionally, such embodiments may include a retroviral vector such as, but not limited to, the MFG or pLJ vectors. An MFG vector is a simplified Moloney murine leukemia virus vector (MoMLV) in which the DNA sequences encoding the pol and env proteins have been deleted to render it replication defective. A pLJ retroviral vector is also a form of the MoMLV (see, e.g., Korman et al. (1987), Proc. Nat'l Acad. Sci., 84:2150-2154). In other embodiments, a recombinant adenovirus or adeno-associated virus can be used as a delivery vector. In other embodiments, the delivery of a recombinant nuclease protein and/or recombinant nuclease gene sequence to a target cell is accomplished by the use of liposomes. The production of liposomes containing nucleic acid and/or protein cargo is known in the art (See, e.g., Lasic et al. (1995), Science 267: 1275-76) Immunoliposomes incorporate antibodies against cell-associated antigens into liposomes and can deliver DNA or mRNA sequences for the meganuclease or the meganuclease itself to specific cell types (see, e.g., Lasic et al. (1995), Science 267: 1275-76; Young et al. (2005), J. Calif. Dent. Assoc. 33(12): 967-71; and Pfeiffer et al. (2006), J. Vasc. Surg. 43(5):1021-7). Methods for producing and using liposome formulations are well known in the art (See, e.g., U.S. Pat. Nos. 6,316,024, 6,379,699, 6,387,397, 6,511,676, and 6,593,308, and references cited therein). In some embodiments, liposomes are used to deliver the sequence of interest as well as the recombinant meganuclease protein or recombinant meganuclease gene sequence.

In certain embodiments, expression of the gene editing nuclease is directly or indirectly controlled by a regulatable promoter or transcription factors activated by an exogenous agent (e.g., a pharmaceutical composition). In alternative embodiments, physiological cues control a regulatable promoter or transcription factors to induce expression of the gene editing nuclease. Promoter systems that are non-leaky and that can be tightly controlled are preferred. Examples of regulatable promoters which are ligand-dependent transcription factor complexes that may be used include, without limitation, members of the nuclear receptor superfamily, which are activated by their respective ligands (e.g., glucocorticoid, estrogen, progestin, retinoid, ecdysone, and analogs and mimetics thereof) and rTTA, which is activated by tetracycline. In certain embodiments, the gene switch is an EcR-based gene switch. Examples of such systems include, without limitation, the systems described in U.S. Pat. Nos. 6,258,603 and 7,045,315, US Published Patent Application Nos. 2006/0014711 and 2007/0161086, and International Publication No. WO 01/70816. Examples of chimeric ecdysone receptor systems are described in U.S. Pat. No. 7,091,038, U.S. Published Patent Application Nos. 2002/0110861, 2004/0033600, 2004/0096942, 2005/0266457, and 2006/0100416, and International Publication Nos. WO 01/70816, WO 02/066612, WO 02/066613, WO 02/066614, WO 02/066615, WO 02/29075, and WO 2005/108617, each of which is incorporated by reference in its entirety. An example of a non-steroidal ecdysone agonist-regulated system is the RheoSwitch® Mammalian Inducible Expression System (New England Biolabs, Ipswich, Mass.).

Still other promoter systems may include response elements such as, but not limited to, a tetracycline (tet) response element (described by Gossen & Bujard, 1992, Proc. Natl. Acad. Sci. USA 89:5547-551), a hormone response element (see, e.g., Lee et al., 1981, Nature 294:228-232; Hynes et al., 1981, Proc. Natl. Acad. Sci. USA 78:2038-2042; Klock et al., 1987, Nature 329:734-736; and Israel & Kaufman, 1989, Nucl. Acids Res. 17:2589-2604), or other inducible promoters known in the art. Using such promoters, expression of the gene editing nuclease and, optionally, other proteins can be controlled, for example, by the Tet-on/off system (Gossen et al., 1995, Science 268:1766-9; Gossen et al., 1992, Proc. Natl. Acad. Sci. USA., 89(12):5547-51); the TetR-KRAB system (Urrutia R., 2003, Genome Biol., 4(10):231; Deuschle U et al., 1995, Mol Cell Biol. (4):1907-14); the mifepristone (RU486) regulatable system (Geneswitch; Wang Y et al., 1994, Proc. Natl. Acad. Sci. USA., 91(17):8180-4; Schillinger et al., 2005, Proc. Natl. Acad. Sci. USA. 102(39):13789-94); the humanized tamoxifen-dep regulatable system (Roscilli et al., 2002, Mol. Ther. 6(5):653-63). The gene switch may be based on heterodimerization of FK506 binding protein (FKBP) with FKBP rapamycin associated protein (FRAP) and be regulated through rapamycin or its non-immunosuppressive analogs. Examples of such systems, include, without limitation, the ARGENT™ Transcriptional Technology (ARIAD Pharmaceuticals, Cambridge, Mass.) and the systems described in U.S. Pat. Nos. 6,015,709, 6,117,680, 6,479,653, 6,187,757, 6,649,595, 5,834,266, 7,109,317, 7,485,441, 5,830,462, 5,869,337, 5,871,753, 6,011,018, 6,043,082, 6,046,047, 6,063,625, 6,140,120, 6,165,787, 6,972,193, 6,326,166, 7,008,780, 6,133,456, 6,150,527, 6,506,379, 6,258,823, 6,693,189, 6,127,521, 6,150,137, 6,464,974, 6,509,152, 6,015,709, 6,117,680, 6,479,653, 6,187,757, 6,649,595, 6,984,635, 7,067,526, 7,196,192, 6,476,200, and 6,492,106, US Published Patent Application Nos. 2002/0173474 and 2009/10100535, International Publication Nos. WO 94/18347, WO 96/20951, WO 96/06097, WO 97/31898, WO 96/41865, WO 98/02441, WO 95/33052, WO 99110508, WO 99110510, WO 99/36553, WO 99/41258, and WO 01114387, ARGENT™ Regulated Transcription Retrovirus Kit, Version 2.0 (9109102), and ARGENT™ Regulated Transcription Plasmid Kit, Version 2.0 (9109/02), each of which is incorporated herein by reference in its entirety. The Ariad system is designed to be induced by rapamycin and analogs thereof, also referred to as “rapalogs”. Examples of suitable rapamycin analogs are provided in the documents listed above in connection with the description of the ARGENT™ system. In certain embodiment, the molecule is rapamycin [e.g., marketed as Rapamune™ by Pfizer]. In another embodiment, a rapalog known as AP21967 [ARIAD] is used. Examples of these dimerizer molecules that can be used in the present invention include, but are not limited to rapamycin, FK506, FK1012 (a homodimer of FK506), and rapamycin analogs (“rapalogs”) which are readily prepared by chemical modifications of the natural product to add a “bump” that reduces or eliminates affinity for endogenous FKBP and/or FRAP. In certain embodiments, a FRAP mutant, such as FRAP-L may be selected. Examples of rapalogs include, but are not limited to, AP26113 (Ariad), AP1510 (Amara, J. F., et al., 1997, Proc Natl Acad Sci USA, 94(20): 10618-23), AP22660, AP22594, AP21370, AP22594, AP23054, AP1855, AP1856, AP1701, AP1861, AP1692 and AP1889, with designed ‘bumps’ that minimize interactions with endogenous FKBP. Still other rapalogs may be selected, such as AP23573 (Merck).

See, also, A V Bruter et al., Molecular Biology, May 2013, Vol 47, Issue 3, pp. 321-342, Naidoo and Young, Neurology Research International, Vol. 2012; Article ID 595410; S. Goverdhana et al, Mol Ther, August 2005; 12(2): 189-211, for discussion of exogenously regulatable promoter systems that may be used in certain embodiments.

The DNA binding domain fusion protein and activation domain fusion protein encoded by the dimerizable fusion proteins may contain one or more copies of one or more different dimerizer binding domains. The dimerizer binding domains may be N-terminal, C-terminal, or interspersed with respect to the DNA binding domain and activation domain. Embodiments involving multiple copies of a dimerizer binding domain usually have 2, 3 or 4 such copies. The various domains of the fusion proteins are optionally separated by linking peptide regions, which may be derived from one of the adjacent domains or may be heterologous.

In certain embodiments, an amount of a pharmaceutical composition comprising a dimerizer (e.g., a rapamycin or rapalog) is administered that is in the range of about 0.1-5 micrograms (μg)/kilogram (kg). To this end, a pharmaceutical composition comprising a dimerizer is formulated in doses in the range of about 7 mg to about 350 mg to treat an average subject of 70 kg in body weight. In certain embodiments, the amount of a pharmaceutical composition comprising a dimerizer administered is: 0.1, 0.2, 0.3, 0.4, 0.5, 0.6, 0.7, 0.8, 0.9, 1.0, 1.5, 2.0, 2.5, 3.0, 3.5, 4.0, 4.5 or 5.0 mg/kg. In certain embodiments, the dose of a dimerizer in a formulation is 7, 8, 9, 10, 20, 25, 30, 35, 40, 45, 50, 55, 60, 65, 70, 75, 80, 85 90, 95, 100, 125, 150, 175, 200, 225, 250, 275, 300, 325, 350, 375, 400, 400, 425, 450, 475, 500, 525, 550, 575, 600, 625, 650, 675, 700, 725, or 750 mg (to treat an average subject of 70 kg in body weight). These doses are preferably administered orally. These doses can be given once or repeatedly, such as daily, every other day, weekly, biweekly, or monthly. Preferably, the pharmaceutical compositions are given once weekly for a period of about 4-6 weeks. In some embodiments, a pharmaceutical composition comprising a dimerizer is administered to a subject in one dose, or in two doses, or in three doses, or in four doses, or in five doses, or in six doses or more. In some embodiments, daily dosages of a pharmaceutical composition comprising a dimerizer may be administered. In other embodiments, weekly dosages of a pharmaceutical composition comprising a dimerizer may be administered.

### Regulatable Genome Editing Compositions

The regulatable systems described herein may be delivered by any suitable route, including non-viral delivery methods or viral delivery methods, in order to treat a disorder associated with a genetic abnormality. A “genetic disorder” is used throughout to refer to any diseases, disorders, or conditions associated with an insertion, change, or deletion in the amino acid sequence of the wild-type protein. Unless otherwise specified, such disorders include inherited and/or non-inherited genetic disorders, as well as diseases and conditions which may not manifest physical symptoms during infancy or childhood.

In either non-viral or viral systems, the genome editing nuclease is expressed in vivo and is under the control of a regulatable promoter, which controls the timing of expression. In certain embodiments, the regulatable system also controls the level of expression, thus allowing the clinician to control the amount of genome editing by controlling the dose of the regulating agent. In certain embodiments, the regulatable system has a regulating agent with a predetermined half-life, thus allowing the clinician to induce expression, remove the agent to provide for an interim period with no expression, and to re-induce expression by reintroducing the regulating agent. One suitable system described herein includes the ARGENT™ system, which may be regulated with a suitable dose of a rapalog.

As provided herein, the minimum components of a composition include, at a minimum: (a) a coding sequence for a gene editing nuclease, and (b) a donor sequence to be inserted into the host cell genome. In certain embodiments, the nuclease is directly under the control of the regulatable promoter. In other embodiments, the nuclease is expressed following activation of a dimerizable DNA binding domain which is under the control of a regulatable promoter. In such embodiments, expression of the activation domain (fusion) protein is typically under the control of a constitutive promoter. In a particularly desirable embodiment, the activation domain fusion protein is under the control of a promoter specific for the tissue (cell) to which the donor sequence is targeted. For example, for liver-targeted donor sequence, a liver-specific promoter may be selected. Liver-specific promoters that may be used [see, e.g., The Liver Specific Gene Promoter Database, Cold Spring Harbor, http://rulai.cshl.edu/LSPD/), include, but are not limited to, alpha 1 anti-trypsin (A1AT), human albumin (Miyatake et al., J. Virol., 71:5124 32 (1997)), humAlb promoter, hepatitis B virus core promoter (Sandig et al., Gene Ther., 3:1002 9 (1996)), TTR minimal enhancer/promoter, alpha-antitrypsin promoter, or LSP (845 nt). For other targets, appropriate tissue-specific promoters may be selected. Other suitable targets may include any cell type, such as, but not limited to, epithelial cells (gut, lung, retina, etc.), central nervous system (CNS) progenitor cells, muscle cells (including, e.g., smooth muscle, cardiac muscle, striated muscle, skeletal muscle). Examples of promoters specific for endothelial cells include, but are not limited to, endothelin-I (ET-I), Flt-I, FoxJ1 (ciliated cells), and T3^{b} [H Aihara et al, FEBS Letters, Vol. 463 (Issues 1-2), p. 185-188 (10 Dec. 1999)] (intestinal epithelial cells), E-cadherin promoter [J. Behrens et al, Proc Natl Acad Sci USA, Vol. 88: 11495-11499 (December 1991)], CEA promoter. Examples of neuron-specific promoters include, e.g., synapsin I (SYN), calcium/calmodulin-dependent protein kinase III, tubulin alpha I, microtubulin-associated protein 1B (MAP1B), neuron-specific enolase (Andersen et al., Cell. Mol Neurobiol., 13:503-15 (1993)), platelet-derived growth factor beta chain, neurofilament light-chain (Piccioli et al., Proc. Natl. Acad. Sci. USA, 88:5611-5 (1991)), neuron-specific vgf (Piccioli et al., Neuron, 15:373-84 (1995)), neuronal nuclei (NeuN), glial fibrillary acidic protein (GFAP), adenomatous polyposis coli (APC), and ionized calcium-binding adapter molecule 1 (Iba-1) gene promoters, and the minimal promoter for HB9 [S Pfaff, Neuron (1999) 23: 675-687; Nature Genetics (1999) 23: 71-75]. In certain embodiments, constitutive promoters may be used.

“Virus stocks” or “stocks of replication-defective virus” refers to viral vectors that package the same artificial/synthetic genome (in other words, a homogeneous or clonal population).

The dual vector system provided herein utilizes a combination of two or more different vector stocks co-administered to a subject. These vectors may be formulated together or separately and delivered essentially simultaneously, preferably by the same route. While the following discussion focuses on AAV vectors, it will be understood that a different, partially or wholly integrating virus (e.g., another parvovirus or a lentivirus) may be used in the system in place of the gene editing vector and/or the vector carrying template.

In one example, the dual vector system comprises (a) a gene editing vector which comprises a gene for an editing enzyme under the control of a regulatable promoter which directs its expression in a target cell (e.g., a hepatocyte) comprising a targeted gene which has one or more mutations resulting in a disorder (e.g., a liver metabolic disease) and (b) a targeting vector comprising a sequence specifically recognized by the editing enzyme and a donor template, wherein the donor template comprises a nucleic acid sequence which replaces at least one of the mutations in the targeted gene.

In certain embodiment, the gene editing vector comprises a Cas9 gene as the editing enzyme and the targeting vector comprises a sgRNA (or “gRNA”) which is at least 20 nucleotides in length and specifically binds to a selected site in the targeted gene and is 5′ to a protospacer-adjacent motif (PAM) which is specifically recognized by the Cas9. Typically, the PAM sequence to the corresponding sgRNA is mutated on the donor template. However, in certain embodiments, the gene editing vector may contain a different Crispr.

“Cas9” (CRISPR associated protein 9) refers to family of RNA-guided DNA endonucleases which is characterized by two signature nuclease domains, RuvC (cleaves non-coding strand) and HNH (coding strand). Suitable bacterial sources of Cas9 include Staphylococcus aureus (SaCas9), Staphylococcus pyogenes (SpCas9), and Neisseria meningitides [K M Estelt et al, Nat Meth, 10: 1116-1121 (2013)]. The wild-type coding sequences may be utilized in the constructs described herein. Alternatively, these bacterial codons are optimized for expression in humans, e.g., using any of a variety of known human codon optimizing algorithms. Alternatively, these sequences may be produced synthetically, either in full or in part. In the examples below, the Staphylococcus aureus (SaCas9) and the Staphylococcus pyogenes (SpCas9) versions of Cas9 were compared. SaCas9 has a shorter sequence. Other endonucleases with similar properties may optionally be substituted (See, e.g., the public CRISPR database (db) accessible at http://crispr.u-psud.fr/crispr).

In another embodiment, the CRISPR system selected may be Cpf1 (CRISPR from Prevotella and Francisella), which may be substituted for a Class 2 CRISPR, type II Cas9-based system in the methods described herein. Cpf1's preferred PAM is 5′-TTN—this contrasts with that of SpCas9 (5′-NGG) and SaCas9 (5′-NNGRRT; N=any nucleotide; R=adenine or guanine) in both genomic location and GC-content. While at least 16 Cpf1 nuclease have been identified, two humanized nucleases (AsCpf1 and LbCpf1) are particularly useful (See http://www.addgene.org/69982/sequences/#depositor-full (AsCpf1 sequences) and http://www.addgene.org/69988/sequences/#depositor-full (LbCpf1 sequences), which are incorporated herein by reference). Further, Cpf1 does not require a tracrRNA, allowing for the use of shorter guide RNAs (about 42 nucleotides) compared to Cas9. Plasmids for various CRISPR systems may be obtained from Addgene, a public plasmid database.

While the CRISPR system can be effective if the ratio of gene editing vector to template vector is about 1 to about 1, it is often desirable for the template vector to be present in excess of the gene editing vector. In certain embodiments, the ratio of editing vector (a) to targeting vector (b) is about 1:3 to about 1:100, or about 1:10. This ratio of gene editing enzyme (e.g., Cas9 or Cpf) to donor template may be maintained even if the enzyme is additionally or alternatively supplied by a source other than the AAV vector. Such embodiments are discussed in more detail below.

In certain embodiments, the gene editing vector includes enhancer elements. Suitable enhancers include, but are not limited to, the alpha fetoprotein enhancer, the TTR minimal promoter/enhancer, LSP (TH-binding globulin promoter/alpha1-microglobulin/bikunin enhancer). Yet other promoters and enhancers can be used to target liver and/or other tissues. Other suitable vector elements may also be included in the gene editing vector. However, the size of the enzyme (Cas9 or Cpf1) gene and packaging limitations of AAV does make it desirable to select truncated or shortened versions of such elements. Thus, while conventional polyA sequences may be selected, including, e.g., SV40 and bovine growth hormone (bGH), shortened and/or synthetic polyAs may also be desired.

In addition to the gene editing vector, the dual AAV vector system utilizes a second type of vector which is an AAV targeting vector comprising a sgRNA and a donor template. Optionally, more than one sgRNA can be used to improve the rates of gene correction. The term “sgRNA” refers to a “single-guide RNA”. sgRNA has at least a 20 base sequence (or about 24-28 bases) for specific DNA binding (homologous to the target DNA). Transcription of sgRNAs should start precisely at its 5′ end. When targeting the template DNA strand, the base-pairing region of the sgRNA has the same sequence identity as the transcribed sequence. When targeting the nontemplate DNA strand, the base-pairing region of the sgRNA is the reverse-complement of the transcribed sequence. Optionally, the targeting vector may contain more than one sgRNA. The sgRNA is 5′ to a protospacer-adjacent motif (PAM) which is specifically recognized by the Cas9 (or Cpf1) enzyme. Typically, the sgRNA is immediately 5′ to the PAM sequence, i.e., there are no spacer or intervening sequences. Examples of sgRNA and PAM sequences designed for correcting a mutation in the OTC gene which causes OTC deficiency are illustrated below. More particularly, the target sequences are designed to correct the G/A mutation associated with OTC deficiency in the position corresponding to nt 243 of wildtype OTC by inserting (or knocking-in) a fragment containing the correct sequence [see, e.g., Genbank entry D00230.2, for genomic DNA sequence and identification of introns and exons, www.ncbi nlm nih.gov/nuccore/-D00230.2].

Typically, the guide RNA may be expressed under the control of a ubiquitous promoter (e.g., a polIII promoter) such as those known in the art. However, in certain embodiments, a tissue-specific promoter (e.g., a polII promoter) or a regulatable promoter such as described herein, is employed. Such promoters are useful in reducing off-target expression of the guide RNA. In certain embodiments, this may be combined with a regulatable promoter for the Cas9 or Cpf1 enzyme.

Suitable tissue-specific promoters may be selected by one of skill in the art based on the target tissue. For example, liver-specific promoters may be used [see, e.g., The Liver Specific Gene Promoter Database, Cold Spring Harbor, http://rulai.schl.edu/LSPD] including, but not limited to, the thyroxine-binding globulin (TBG) promoter, alpha 1 anti-trypsin (A1AT) promoter, human albumin (humAlb) promoter [Miyatake et al., J. Virol., 71:5124 32 (1997)], hepatitis B virus core promoter [Sandig et al., Gene Ther., 3:1002 9 (1996)], TTR minimal enhancer/promoter, alpha-antitrypsin promoter, and LSP (845 nt). For a different target tissue (e.g., epithelial or CNS cells), a different tissue-specific promoter may be selected. Examples of promoters specific for endothelial cells include, but are not limited to, endothelin-I (ET-I), Flt-I, FoxJ1 (for targeting ciliated cells), and T3^{b} [H Aihara et al, FEBS Letters, Vol. 463 (Issues 1-2), p. 185-188 (10 Dec. 1999) (for targeting intestinal epithelial cells), E-cadherin promoter (J. Behrens et al, Proc Natl Acad Sci USA, Vol. 88: 11495-11499 (December 1991)], and CEA promoter. Examples of neuron-specific promoters include, e.g., synapsin I (SYN), calcium/calmodulin-dependent protein kinase III, tubulin alpha I, microtubulin-associated protein 1B (MAP1B), neuron-specific enolase (Andersen et al., Cell. Mol. Neurobiol., 13:503-15 (1993)), platelet-derived growth factor beta chain promoters, neurofilament light-chain gene (Piccioli et al., Proc. Natl. Acad. Sci. USA, 88:5611-5 (1991)), neuron-specific vgf gene (Piccioli et al., Neuron, 15:373-84 (1995)), neuronal nuclei (NeuN), glial fibrillary acidic protein (GFAP), adenomatous polyposis coli (APC), and ionized calcium-binding adapter molecule 1 (Iba-1) promoters, or the minimal promoter for HB9 [S Pfaff, Neuron (1999) 23: 675-687; Nature Genetics (1999) 23: 71-75]. Examples of suitable exogenously regulatable promoter systems are described elsewhere in this specification and are incorporated herein by reference (See, also, AV Bruter et al, Molecular Biology, May 2013, Vol 47, Issue 3, pp. 321-342, Naidoo and Young, Neurology Research International, Vol. 2012; Article ID 595410; S. Goverdhana et al, Mol Ther, August 2005; 12(2): 189-211).

In general, a PAM sequence for SaCas9 has an NNGRRT motif. Once a selected target sequence is selected, an sgRNA comprising the target and PAM sequence may be generated synthetically, or using conventional site-directed mutagenesis. In the examples below illustrating correction of the ornithine transcarbamylase (OTC) gene, the target DNA is within intron 4, which is 3′ to the G/A mutation site. However, other suitable target sites may be selected for other mutations targeted for correction (See, e.g., http://omim.org/entry/311250). The target sites are typically selected such that they do not disrupt expression of functional portions of the gene. Optionally, more than one correction may be made to a target gene using the system described herein. Suitably, the vectors delivering donor template which are gene fragments are designed such that the donor template is inserted upstream of the gene mutation or phenotype to be corrected.

In certain embodiments, a full-length functioning gene may be inserted into the genome to replace the defective gene. Thus, in certain embodiments, the inserted sequence may be a full-length gene, or a gene encoding a functional protein or enzyme. Where a full-length gene is being delivered, there is more flexibility within the target gene for targeting. In an alternative embodiment, a single exon may be inserted upstream of the defective exon. In yet another embodiment, gene deletion or insertion is corrected.

In still another embodiment, the compositions described herein are used to reduce expression of a gene having undesirably high expression levels. Such a gene may be a PCSK9 which binds to the receptor for low-density lipoprotein (LDL) cholesterol; reducing PCSK9 expression can be used to increase circulating LDL cholesterol levels. In other embodiments, the composition targets a cancer-associated genes (e.g., BRCA1 or BRCA2) (See also, http://www.eupedia.com/genetics/cancer_related_snp.shtml).

A variety of different AAV capsids have been described and may be used, although AAV which preferentially target the liver and/or deliver genes with high efficiency are particularly desired. The sequences of AAV8 (and other AAV members of Glade E) have been previously described (available in U.S. Pat. Nos. 7,790,449 and 7,282,199, and in a variety of public databases). While the examples utilize AAV vectors having the same capsid, the capsid of the gene editing vector and the targeting vector may or may not be the same AAV capsid. Another suitable AAV may be used, e.g., rh10 (WO 2003/042397). Still other suitable AAV vectors include, e.g., AAV9 (U.S. Pat. No. 7,906,111; US 2011-0236353-A1), hu37 (see, e.g., U.S. Pat. No. 7,906,111; US 2011-0236353-A1), AAV1, AAV2, AAV3, AAV4, AAV5, AAV6, AAV6.2, AAV7, AAV8 (U.S. Pat. Nos. 7,790,449 and 7,282,199 and others). See, e.g., WO 2003/042397, WO 2005/033321, WO 2006/110689, U.S. Pat. Nos. 7,790,449, 7,282,199, and 7,588,772B2 for sequences of these and other suitable AAV, as well as methods for generating AAV vectors. Still other AAV may be selected, optionally taking into consideration tissue preferences of the selected AAV capsid.

A “recombinant AAV” or “rAAV” is a DNAse-resistant viral particle containing two elements, an AAV capsid and a vector genome containing at least non-AAV coding sequences packaged within the AAV capsid. Unless otherwise specified, this term may be used interchangeably with the phrase “rAAV vector”.

As used herein, a “vector genome” refers to the nucleic acid sequence packaged inside a vector capsid. The vector genome is composed of, at a minimum, a transgene and its regulatory sequences, and 5′ and 3′ AAV inverted terminal repeats (ITRs). It is this vector genome which is packaged into a capsid and delivered to a selected target cell or target tissue. A recombinant AAV vector may comprise, packaged within an AAV capsid, a nucleic acid molecule containing a 5′ AAV ITR, the expression cassettes described herein and a 3′ AAV ITR. As described herein, an expression cassette may contain regulatory elements for an open reading frame(s) within each expression cassette and the nucleic acid molecule may optionally contain additional regulatory elements.

Where a pseudotyped AAV is to be produced, the ITRs are selected from a source which differs from the AAV source of the capsid. For example, AAV2 ITRs may be selected for use with an AAV capsid having a particular efficiency for a selected cellular receptor, target tissue, or viral target. In certain embodiments, the ITR sequences from AAV2, or the deleted version thereof (ΔITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. However, other sources of AAV ITRs may be utilized.

The AAV vector genome may contain a full-length AAV 5′ inverted terminal repeat (ITR) and a full-length 3′ ITR. A shortened version of the 5′ ITR, termed ΔITR, has been described in which the D-sequence and terminal resolution site (trs) are deleted. The abbreviation “sc” refers to self-complementary. “Self-complementary AAV” refers to a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription (See, e.g., DM McCarty et al., “Self-complementary recombinant adeno-associated virus (scAAV) vectors promote efficient transduction independently of DNA synthesis”, Gene Therapy, (August 2001), Vol 8, Number 16, Pages 1248-1254). Self-complementary AAVs are also described in, e.g., U.S. Pat. Nos. 6,596,535, 7,125,717, and 7,456,683, each of which is incorporated herein by reference in its entirety.

The AAV sequences of the vector genome typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In certain embodiment, the ITRs are from an AAV different than that supplying a capsid, resulting in a pseudotyped vector. In certain embodiments, the ITR sequences from AAV2. A shortened version of the 5′ ITR, termed ΔITR, has been described in which the D-sequence and terminal resolution site (trs) are deleted. In other embodiments, the full-length AAV 5′ and 3′ ITRs are used. However, ITRs from other AAV sources may be selected. Where the source of the ITRs is from AAV2 and the AAV capsid is from another AAV source, the resulting vector may be termed pseudotyped. However, other configurations of these elements may be suitable.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus.

As used herein, “operably linked” sequences include both expression control sequences that are contiguous with the gene of interest and expression control sequences that act in trans or at a distance to control the gene of interest.

A single-stranded AAV viral vector may be used. Methods for generating and isolating AAV viral vectors suitable for delivery to a subject are known in the art (See, e.g., U.S. Pat. Nos. 7,790,44, 7,282,199, and 7,588,772 B2 and International Publication Nos. WO 2003/042397, WO 2005/033321, WO 2006/110689). In one system, a producer cell line is transiently transfected with a construct that encodes the transgene flanked by ITRs and a construct(s) that encodes rep and cap. In a second system, a packaging cell line that stably supplies rep and cap is transfected (transiently or stably) with a construct encoding the transgene flanked by ITRs. In each of these systems, AAV virions are produced in response to infection with helper adenovirus, herpesvirus, or baculovirus, requiring the separation of the rAAVs from contaminating virus. More recently, systems have been developed that do not require infection with helper virus to recover the AAV—the required helper functions (i.e., adenovirus E1, E2a, VA, and E4, herpesvirus ULS, ULB, UL52, and UL29 and herpesvirus polymerase; or baculovirus) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For a review on these production systems, see Zhang et al., 2009, “Adenovirus-adeno-associated virus hybrid for large-scale recombinant adeno-associated virus production,” Human Gene Therapy 20:922-929, the contents which is incorporated herein by reference in its entirety. Methods of making and using these and other AAV production systems are also described in the following US patents, the contents of which are incorporated herein by reference in their entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The rAAV may be generated using methods described herein, or other methods described in the art, and purified as described. See, e.g., M. Mietzsch et al., “OneBac: Platform for Scalable and High-Titer Production of Adeno-Associated Virus Serotype 1-12 Vectors for Gene Therapy, Hum Gene Ther. 2014 Mar. 1; 25(3): 212-222. See, also, Smith R H, et al, Mol Ther, 2009 November; 17(11): 1888-96 (2009), describing a simplified baculovirus-AAV vector expression system coupled with one-step affinity purification. For example, lysates or supernatants (e.g., treated, freeze-thaw supernatants or media containing secreted rAAV), may be purified using one-step AVB sepharose affinity chromatography using 1 ml prepacked HiTrap columns on an ACTA purifier (GE Healthcare) as described by manufacturer, or in M. Mietzsch, et al., cited above. In one embodiment, an affinity capture method is performed using an antibody-capture affinity resin. See, e.g. WO 2017/015102. Alternatively, the rAAV used herein may be purified using other techniques known in the art.

Methods of preparing AAV-based vectors are known. See, e.g., US Published Patent Application No. 2007/0036760 (Feb. 15, 2007), which is incorporated by reference herein. The use of AAV capsids having tropism for muscle cells and/or cardiac cells are particularly well suited for the compositions and methods described herein. However, other targets may be selected. The sequences of AAV9 and methods of generating vectors based on the AAV9 capsid are described in U.S. Pat. No. 7,906,111, US2015/0315612, WO 2012/112832, and WO 2017/160360A3, which are incorporated herein by reference. In certain embodiments, the sequences of AAV1, AAV5, AAV6, AAV9, AAV8triple, Anc80, Anc81 and Anc82 are known and may be used to generate AAV vector. See, e.g., U.S. Pat. No. 7,186,552, WO 2017/180854, U.S. Pat. No. 7,282,199 B2, U.S. Pat. Nos. 7,790,449, and 8,318,480, which are incorporated herein by reference. The sequences of a number of such AAV are provided in the above-cited U.S. Pat. No. 7,282,199 B2, U.S. Pat. Nos. 7,790,449, 8,318,480, 7,906,111, WO 2003/042397, WO 2005/033321, WO 2006/110689, U.S. Pat. Nos. 8,927,514, 8,734,809; WO 2015054653A3, WO 2016/065001-A1, WO 2016/172008-A1, WO 2015/164786-A1, US 2010/186103-A1, WO-010/138263-A2, and WO 2016/049230A1, and/or are available from GenBank. Corresponding methods have been described for AAV1, AAV8, and AAVrh10-like vectors. See, WO 2017/100676 A1, WO 2017/100674 A1, and WO 2017/100704 A1.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid; a functional rep gene; an expression cassette composed of, at a minimum, AAV inverted terminal repeats (ITRs) and a transgene; and sufficient helper functions to permit packaging of the expression cassette into the AAV capsid protein. The host cell may be a 293 cell or a suspension 293 cell. See, e.g., Zinn, E., et al., as cited herein; Joshua C Grieger et al. Production of Recombinant Adeno-associated Virus Vectors Using Suspension HEK293 Cells and Continuous Harvest of Vector From the Culture Media for GMP FIX and FLT1 Clinical Vector. Mol Ther. 2016 February; 24(2): 287-297. Published online 2015 Nov. 3. Prepublished online 2015 Oct. 6. doi: 10.1038/mt.2015.187; Laura Adamson-Small, et al. Sodium Chloride Enhances Recombinant Adeno-Associated Virus Production in a Serum-Free Suspension Manufacturing Platform Using the Herpes Simplex Virus System. Hum Gene Ther Methods. 2017 Feb. 1; 28(1): 1-14. Published online 2017 Feb. 1. doi: 10.1089/hgtb.2016.151; US20160222356A1; and Chahal P S et al. Production of adeno-associated virus (AAV) serotypes by transient transfection of HEK293 cell suspension cultures for gene delivery. J Virol Methods. 2014 February; 196:163-73. doi: 10.1016/j.jviromet.2013.10.038. Epub 2013 Nov. 13.

Other methods of producing rAAV available to one of skill in the art may be utilized. Suitable methods may include without limitation, baculovirus expression system (e.g., baculovirus-infected-insect-cell system) or production via yeast. See, e.g., WO 2005/072364A2; WO 2007/084773 A2; WO 2007/148971A8; WO 2017/184879A1; WO 2014/125101A1; U.S. Pat. No. 6,723,551 B2; Bryant, L. M., et al., Lessons Learned from the Clinical Development and Market Authorization of Glybera. Hum Gene Ther Clin Dev, 2013; Robert M. Kotin, Large-scale recombinant adeno-associated virus production. Hum Mol Genet. 2011 Apr. 15; 20(R1): R2-R6. Published online 2011 Apr. 29. doi: 10.1093/hmg/ddr141; Aucoin M G et al., Production of adeno-associated viral vectors in insect cells using triple infection: optimization of baculovirus concentration ratios. Biotechnol Bioeng. 2006 Dec. 20; 95(6):1081-92; Sami S. Thakur, Production of Recombinant Adeno-associated viral vectors in yeast. Thesis presented to the Graduate School of the University of Florida, 2012; Kondratov O, et al. Direct Head-to-Head Evaluation of Recombinant Adeno-associated Viral Vectors Manufactured in Human versus Insect Cells, Mol Ther. 2017 Aug. 10. pii: S1525-0016(17)30362-3. doi: 10.1016/j.ymthe.2017.08.003. [Epub ahead of print]; Mietzsch M et al, OneBac 2.0: Sf9 Cell Lines for Production of AAV1, AAV2, and AAV8 Vectors with Minimal Encapsidation of Foreign DNA. Hum Gene Ther Methods. 2017 February; 28(1):15-22. doi: 10.1089/hgtb.2016.164; Li, L. et al. Production and characterization of novel recombinant adeno-associated virus replicative-form genomes: a eukaryotic source of DNA for gene transfer. PLoS One. 2013 Aug. 1; 8(8):e69879. doi: 10.1371/journal.pone.0069879. Print 2013; Galibert, L. et al, Latest developments in the large-scale production of adeno-associated virus vectors in insect cells toward the treatment of neuromuscular diseases. J Invertebr Pathol. 2011 July; 107 Suppl:S80-93. doi: 10.1016/j.jip.2011.05.008; and Kotin R M, Large-scale recombinant adeno-associated virus production. Hum Mol Genet. 2011 Apr. 15; 20(R1):R2-6. doi: 10.1093/hmg/ddr141. Epub 2011 Apr. 29.

In another embodiment, other viral vectors may be used, including integrating viruses, e.g., herpesvirus or lentivirus, although other viruses may be selected. Suitably, where one of these other vectors is generated, it is produced as a replication-defective viral vector. A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient (i.e., they cannot generate progeny virions but retain the ability to infect target cells). In certain embodiments, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production.

A variety of different diseases and conditions associated with one or more genetic deletions, insertions, or mutations, may be treated using the methods described herein. Examples of such conditions include, e.g., alpha-1-antitrypsin deficiency, liver conditions such as biliary atresia, Alagille syndrome, alpha-1 antitrypsin, tyrosinemia, neonatal hepatitis, and Wilson disease, metabolic conditions such as biotinidase deficiency, carbohydrate deficient glycoprotein syndrome (CDGS), Crigler-Najjar syndrome, diabetes insipidus, Fabry, galactosemia, glucose-6-phosphate dehydrogenase (G6PD), fatty acid oxidation disorders, glutaric aciduria, hypophosphatemia, Krabbe, lactic acidosis, lysosomal storage diseases, mannosidosis, maple syrup urine, mitochondrial, neuro-metabolic, organic acidemias, PKU, purine, pyruvate dehydrogenase deficiency, urea cycle conditions, vitamin D deficiency, and hyperoxaluria, urea cycle disorders such as N-acetylglutamate synthase deficiency, carbamoyl phosphate synthetase I deficiency, ornithine transcarbamylase deficiency, “AS deficiency” or citrullinemia, “AL deficiency” or argininosuccinic aciduria, and “arginase deficiency” or argininemia.

Other diseases may also be selected for treatment according to the method described herein. Such diseases include, e.g., cystic fibrosis (CF), hemophilia A (associated with defective factor VIII), hemophilia B (associated with defective factor IX), mucopolysaccharidosis (MPS) (e.g., Hunter syndrome, Hurler syndrome, Maroteaux-Lamy syndrome, Sanfilippo syndrome, Scheie syndrome, Morquio syndrome, other, MPSI, MPSII, MPSIII, MSIV, MPS 7), ataxia (e.g., Friedreich ataxia, spinocerebellar ataxias, ataxia telangiectasia, essential tremor, spastic paraplegia), Charcot-Marie-Tooth (e.g., peroneal muscular atrophy, hereditary motor sensory neuropathy), glycogen storage diseases (e.g., type I, glucose-6-phosphatase deficiency, Von Gierke), II (alpha glucosidase deficiency, Pompe), III (debrancher enzyme deficiency, Cori), IV (brancher enzyme deficiency, Anderson), V (muscle glycogen phosphorylase deficiency, McArdle), VII (muscle phosphofructokinase deficiency, Tauri), VI (liver phosphorylase deficiency, Hers), IX (liver glycogen phosphorylase kinase deficiency). This list is not exhaustive and other genetic conditions are identified, e.g., at www.kumc.edu/gec/support; http://www.genome.gov/10001200 and http://www.ncbi.nlm.nih.gov/books/NBK22183/, which are incorporated herein by reference.

Other conditions that may be treated using the methods described herein include central nervous system (CNS)-related disorders. As used herein, a “CNS-related disorder” is a disease or condition of the central nervous system. Such disorders may affect the spinal cord, brain, or tissues surrounding the brain and spinal cord. Non-limiting examples of CNS-related disorders include Parkinson's disease, lysosomal storage Disease, ischemia, neuropathic pain, amyotrophic lateral sclerosis (ALS) (e.g., linked to a mutation in the gene coding for superoxide dismutase, SOD1), multiple sclerosis (MS), and Canavan disease (CD), or a primary or metastatic cancer.

In another embodiment, cells of the retina are targeted, including retinal pigment epithelium (RPE) and photoreceptors, e.g., for treatment of retinitis pigmentosa and/or Leber congenital amaurosis (LCA). Optionally, this treatment may utilize or follow subretinal injection and/or be used in conjunction with the standard of care for the condition.

In one aspect, the method is useful in treating a disorder, comprising: co-administering to a subject having the disorder.

In certain embodiments, the ratio of editing vector to targeting vector is about 1:3 to about 1:100, inclusive of intervening ratios. For example, the ratio of editing vector to targeting vector may be about 1:5 to about 1:50, or about 1:10, or about 1:20. Although not as preferred, the ratio may be 1:1 or there may be more targeting vector.

In general, the ratio of AAV vectors is determined based on particle copies (pt) or genome copies (GC), which terms may be used interchangeably herein, for each vector. Suitably, when determining the ratio of two or more AAV vectors to one another (e.g., editing vector to targeting vector), the same method is used to determine the number of each type of vector(s). However, if different methods are determined to be substantially equivalent, different techniques may be used. Suitable methods for determining GC have been described and include, e.g., oqPCR or digital droplet PCR (ddPCR) as described in, e.g., M. Lock et al., Hu Gene Therapy Methods, Hum Gene Ther Methods. 2014 April; 25(2):115-25. doi: 10.1089/hgtb.2013.131. Epub 2014 Feb. 14, which is incorporated herein by reference.

The compositions described herein are designed for delivery to subjects in need thereof by any suitable route or a combination of different routes. For treatment of liver disease, direct or intrahepatic delivery to the liver is desired and may optionally be performed via intravascular delivery, e.g., via the portal vein, hepatic vein, bile duct, or by transplant. Alternatively, other routes of administration may be selected such as oral, inhalation, intranasal, intratracheal, intraarterial, intraocular, intravenous, intramuscular, and other parental routes. For example, intravenous delivery may be selected for delivery to proliferating, progenitor, and/or stem cells. Alternatively, another route of delivery may be selected. The delivery constructs described herein may be delivered in a single composition or multiple compositions. Optionally, two or more different AAV may be delivered (See, e.g, WO 2011/126808 and WO 2013/049493). In another embodiment, the dual vector system may contain only a single AAV and a second, different Cas9-delivery system. For example, Cas9 (or Cpf1) delivery may be mediated by non-viral constructs (e.g., “naked DNA”, “naked plasmid DNA”, RNA, or mRNA coupled with a delivery composition or nanoparticle, including, e.g., micelles, liposomes, cationic lipid-nucleic acid compositions, poly-glycan compositions and other polymers, lipid and/or cholesterol-based nucleic acid conjugates, and other constructs such as are described herein (See, e.g., X. Su et al., Mol. Pharmaceutics, 2011, 8 (3), pp 774-787; web publication: Mar. 21, 2011; WO2013/182683, WO 2010/053572, and WO 2012/170930, all of which are incorporated herein by reference). Such non-viral delivery constructs may be administered by the routes described previously.

The viral vectors, or non-viral DNA or RNA transfer moieties can be formulated with a physiologically acceptable carrier for use in gene transfer and gene therapy applications. In the case of AAV viral vectors, quantification of the genome copies (“GC”) may be used as the measure of the dose contained in the formulation. Any method known in the art can be used to determine the GC number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome (usually poly A signal). The replication-defective virus compositions can be formulated in dosage units to contain an amount of replication-defective virus that is in the range of about 1.0×10⁹ GC to about 1.0×10¹⁵ GC (to treat an average subject of 70 kg in body weight), and preferably 1.0×10¹² GC to 1.0×10¹⁴ GC for a human patient. Preferably, the dose of replication-defective virus in the formulation is 1.0×10⁹ GC, 5.0×10⁹ GC, 1.0×10¹⁰ GC, 5.0×10¹⁰ GC, 1.0×10¹¹ GC, 5.0×10¹¹ GC, 1.0×10¹² GC, 5.0×10¹² GC, or 1.0×10¹³ GC, 5.0×10¹³ GC, 1.0×10¹⁴ GC, 5.0×1014 GC, or 1.0×10¹⁵ GC.

Production of lentivirus is measured as described herein and expressed as IU per volume (e.g., mL). IU is infectious unit, or alternatively transduction units (TU); IU and TU can be used interchangeably as a quantitative measure of the titer of a viral vector particle preparation. The lentiviral vector is typically integrating. The amount of viral particles is at least about 3×10⁶ IU, and can be at least about 1×10⁷ IU, at least about 3×10⁷ IU, at least about 1×10⁸ IU, at least about 3×10⁸ IU, at least about 1×10⁹ IU, or at least about 3×10⁹ IU.

In addition, the system described herein may involve co-administration of a nucleic acid molecule via a viral or non-viral system. For example, a Cas9 (or Cpf1) sequence may be delivered via a carrier system for expression or delivery in RNA form (e.g., mRNA) using one of a number of carrier systems which are known in the art. Such carrier systems include those provided by commercial entities, such as PhaseRx′ so-called “SMARTT” technology. These systems utilize block copolymers for delivery to a target host cell. See, e.g., US 2011/0286957 entitled, “Multiblock Polymers”, published Nov. 24, 2011; US 2011/0281354, published Nov. 17, 2011; EP2620161, published Jul. 31, 2013; and WO 2015/017519, published Feb. 5, 2015. See, also, S. Uchida et al, (February 2013) PLoS ONE 8(2): e56220. Still other methods involve generating and injecting synthetic dsRNAs [see Soutschek et al., Nature (2004) 432(7014):173-8 and Morrissey et al., Hepatol. (2005) 41(6):1349-56]. Local administration to the liver has also been demonstrated by injecting double stranded RNA directly into the circulatory system surrounding the liver using renal vein catheterization [See Hamar et al., PNAS (2004) 101(41): 14883-8.]. Still other systems involve delivery of dsRNA and particularly siRNA using cationic complexes or liposomal formulations [see, e.g., Landen et al. Cancer Biol. Ther. (2006) 5(12) and Khoury et al., Arthritis Rheumatol. (2006) 54(6): 1867-77]. Other RNA delivery technologies are also available, e.g., from Veritas Bio [see, e.g., US 2013/0323001, published Dec. 23, 2010, “In vivo delivery of double stranded RNA to a target cell” (cytosolic content including RNAs, e.g., mRNA, expressed siRNA/shRNA/miRNA, as well as injected/introduced siRNA/shRNA/miRNA, or possibly even transfected DNA present in the cytosol packaged within exovesicles and be transported to distal sites such as the liver)]. Still other systems for in vivo delivery of RNA sequences have been described (See, e.g., US 2012/0195917 (Aug. 2, 2012) (5′-cap analogs of RNA to improve stability and increase RNA expression), WO 2013/143555A1, Oct. 3, 2013, and/or are commercially available (BioNTech, Germany; Valera (Cambridge, Mass.); Zata Pharmaceuticals).

DNA and RNA are generally measured in nanogram (ng) to microgram (μg) amounts. In general, for a treatment in a human, preferably dosages of the RNA in the range of 1 ng to 700 μg, 1 ng to 500 μg, 1 ng to 300 μg, 1 ng to 200 μg, or 1 ng to 100 μg are formulated and administered. Similar dosage amounts of a DNA molecule (e.g., containing a Cas9 or other expression cassette) not delivered to a subject via a viral vector may be utilized for non-viral DNA delivery constructs.

The above-described recombinant vectors or other constructs may be delivered to host cells according to published methods. The vectors or other moieties are preferably suspended in a physiologically compatible carrier, may be administered to a human or non-human mammalian patient. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the nucleic acid molecules (or vectors carrying same) and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The system described herein may be therapeutically useful if a sufficient amount of functional enzyme or protein is generated to improve the patient's condition. In certain embodiments, gene expression levels as low as 5% of healthy patients will provide sufficient therapeutic effect for the patient to be treatable by gene therapy approaches. In other embodiments, gene expression levels are at least about 10%, at least about 15% to up to 100% of the normal range (levels) observed in humans (or veterinary subject). “Functional enzyme” is meant to refer to a gene which encodes the wild-type enzyme (e.g., OTCase) which provides at least about 50%, at least about 75%, at least about 80%, at least about 90%, or about the same, or greater than 100% of the biological activity level of the wild-type enzyme, or a natural variant or polymorph thereof which is not associated with disease. More particularly, as heterozygous patients may have as low an enzyme functional level as about 50% or lower, effective treatment may not require replacement of enzyme activity to levels within the range of “normal” or non-deficient patients. Similarly, patients having no detectable amounts of enzyme may be rescued by delivering enzyme function to less than 100% activity levels, and may optionally be subject to further treatment subsequently. In certain embodiments, where gene function is being delivered by the donor template, patients may express higher levels than found in “normal”, healthy subjects. In still other embodiments, where reduction in gene expression is desired, as much as a 20% reduction to a 50% reduction, or up to about 100% reduction, may provide desired benefits. As described herein, the therapy described herein may be used in conjunction with other treatments, i.e., the standard of care for the subject's (patient's) diagnosis.

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% (±10%) from the reference given, unless otherwise specified.

A “subject” is a mammal, e.g., a human, mouse, rat, guinea pig, dog, cat, horse, cow, pig, or non-human primate, such as a monkey, chimpanzee, baboon or gorilla. A patient refers to a human A veterinary subject refers to a non-human mammal.

As used herein, “disease”, “disorder”, and “condition” are used interchangeably to indicate an abnormal state in a subject.

Unless defined otherwise in this specification, technical and scientific terms used herein have the same meaning as commonly understood by one of ordinary skill in the art and by reference to published texts, which provide one skilled in the art with a general guide to many of the terms used in the present application.

The following examples are illustrative only and are not a limitation on the invention described herein.

### EXAMPLES

### Example 1: Two Vector System

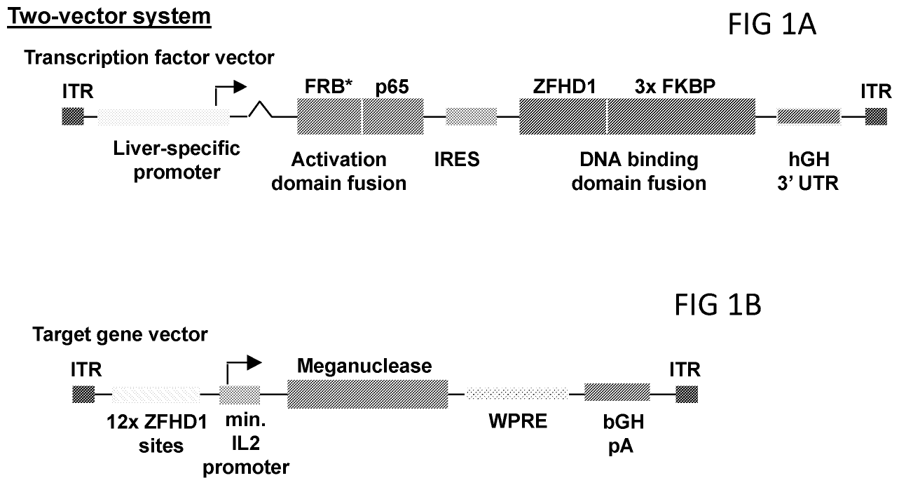

A transcription factor vector is generated which contains in its vector genome, from 5′ to 3′: a 5′-ITR, a liver-specific promoter operably linked to an FRB+p65 activation domain fusion protein, a linker (IRES), a DNA binding domain fusion protein (zinc finger HD1 and three FK binding proteins), and a human growth hormone 3′ UTR, followed by a 3′-ITR (FIG. 1A). A rAAV may be generated by triple transfection using a plasmid expressing a desired AAV capsid such as AAV8 and a plasmid carrying the required rep and/or helper virus sequences required for replication and packaging in a suitable packaging host cell.

The FRB+p65 is a dimerizable transcription factor domain unit (FRB fused with p65 activation domain). The FRB fragment corresponds to amino acids 2021-2113 of FRAP (FKBP rapamycin-associated protein, also known as mTOR [mammalian target of rapamycin]), a phosphoinositide 3-kinase homolog that controls cell growth and division. The FRAP sequence incorporates the single point-mutation Thr2098Leu (FRAPL or FRAP-L) to allow use of certain non-immunosuppressive rapamycin analogs (rapalogs). FRAP binds to rapamycin (or its analogs) and FKBP and is fused to a portion of human NF-KB p65 (190 amino acids) as transcription activator.

ZFHD-FKBP fusion: fusion of a DNA binding domain and 1 copy of a dimerizer binding domain (1×FKBP; 732 bp), 2 copies of drug binding domain (2×FKBP; 1059 bp), or 3 (3×FKBP; 1389 bp) copies of drug binding domain Immunophilin FKBP (FK506-binding protein) is an abundant 12 kDa cytoplasmic protein that acts as the intracellular receptor for the immunosuppressive drugs FK506 and rapamycin. ZFHD is a DNA binding domain composed of a zinc finger pair and a homeodomain. Both fusion proteins contain N-terminal nuclear localization sequences from human c-Myc at the 5′ end.

A second vector for co-administration with transcription factor vector is a target gene vector which contains, from 5′ to 3′: a 5′-ITR, 12 zinc finger HD1 sites, a minimal IL2 promoter operably linked to a meganuclease coding sequence, a woodchuck post-regulatory element (WPRE), a bovine growth hormone polyA (bGH pA), and a 3′-ITR (FIG. 1B). A second rAAV is generated using triple transfection as described for the transcription factor vector. Suitably, the two vectors have the same capsid.

Prior to delivery to a subject, the two vectors may be mixed and administered in the same composition (e.g., injection or infusion). It will be understood that for targeting tissue other than the liver, a different tissue specific promoter is selected and a different capsid may be selected.

### Example 2: Single Vector System

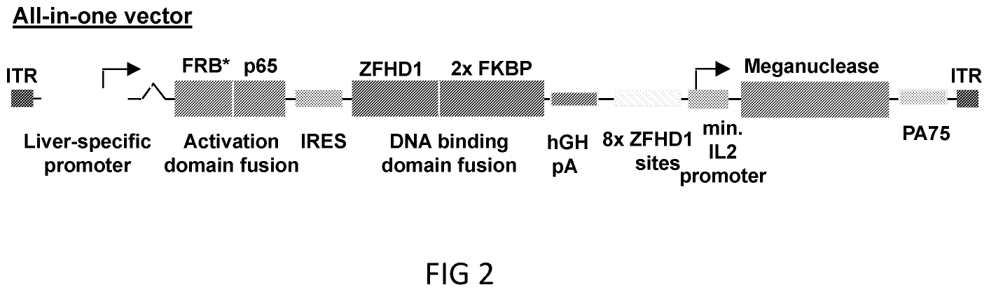

In this system, the transcription factor and the target gene are in a single vector genome. As illustrated in FIG. 2, the genome includes, from 5′ to 3′: an ITR, a liver-specific promoter which directs control of an activation domain fusion protein, a linker, a DNA binding domain fusion, a human GH poly A, eight zinc finger sites, a minimum IL2 promoter operably linked to a meganuclease coding sequence, a polyA, and an ITR. The ZFHD-FKBP fusion includes two copies of the drug binding domain (2×FKBP; 1059 bp) and eight copies of the zinc finger homeodimer. For an AAV vector, the ITRs selected are AAV-ITRs. They may be generated by triple transfection using a plasmid expressing a desired AAV capsid such as AAV8 and a plasmid carrying the required rep and/or helper virus sequences required for replication and packaging in a suitable packaging host cell.

### Example 3: Dual Vector System for CRISPR/Cas

A two-vector system suitable for liver-targeted therapy in which the gene editing nuclease is Cas9 may be prepared as follows.

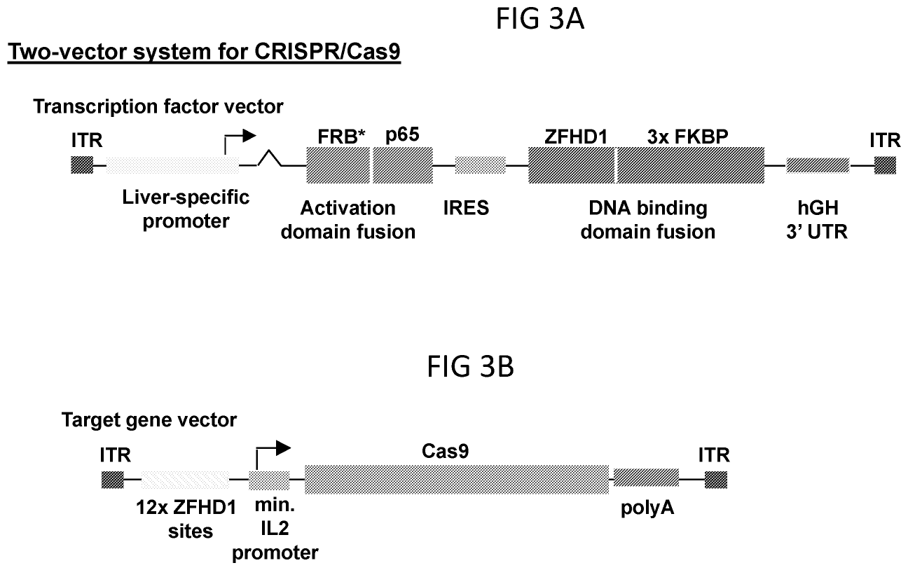

A transcription factor vector is generated which contains in its vector genome, from 5′ to 3′: a 5′-ITR, a liver-specific promoter operably linked to an FRB+p65 activation domain fusion protein, a linker (IRES), a DNA binding domain fusion protein (zinc finger HD1 and three FK binding proteins), and a human growth hormone 3′ UTR, followed by a 3′-ITR (FIG. 3A). A rAAV may be generated by triple transfection using a plasmid expressing a desired AAV capsid such as AAV8 and a plasmid carrying the required rep and/or helper virus sequences required for replication and packaging in a suitable packaging host cell.

A second vector for co-administration with transcription factor vector is a target gene vector which contains, from 5′ to 3′: a 5′-ITR, 12 zinc finger HD1 sites, a minimal IL2 promoter operably linked to a Cas9 coding sequence, a bovine growth hormone polyA (bGH pA), and a 3′-ITR (FIG. 3B). A second rAAV is generated using triple transfection as described for the transcription factor vector. Suitably, the two vectors have the same capsid.

Prior to delivery to a subject, the two vectors may be mixed and administered in the same composition (e.g., injection or infusion). It will be understood that for targeting tissue other than the liver, a different tissue specific promoter and/or a different capsid may be selected.

All publications, patents, and patent applications cited in this application and priority document U.S. Provisional Patent Application No. 62/501,338, filed May 4, 2017, are hereby incorporated by reference in their entireties. Although the foregoing invention has been described in some detail by way of illustration and example for purposes of clarity of understanding, it will be readily apparent to those of ordinary skill in the art in light of the teachings of this invention that certain changes and modifications can be made thereto without departing from the spirit or scope of the appended claims.

1. A method of regulating a gene editing system in vivo, said method comprising delivering to a subject one or more nucleic acid molecules comprising:
(a) at least one nucleic acid sequence encoding one or more DNA binding domains,
(c) at least one nucleic acid sequence comprising a coding sequence of an activation domain for the regulatable promoter;
(c) at least one coding sequence encoding a nuclease; wherein expression of the nuclease is under the control of at least one regulatable promoter, wherein the promoter is activated and/or regulated by an exogenous agent; and
(d) optionally a nucleic acid sequence comprising a donor gene for insertion into a selected gene locus.

2. The method according to claim 1, wherein the gene editing nuclease is selected from a meganuclease, a zinc finger nuclease, a TALEN, or a CRISPR enzyme (such as Cas9 or Cpf1) or their homologs.

3. The method according to claim 2, wherein one or more of the DNA binding domains comprise a fusion protein having a dimerizable DNA binding domain selected from a zinc finger DNA binding domain, a FK506 binding protein (FKBP), an FKBP rapamycin associated protein (FRAP).

4. The method according to claim 2, wherein the activation domain is a FRB-p65 fusion.

5. The method according to claim 1, wherein the exogenous agent is a pharmaceutical composition comprising rapamycin or a rapalog.

6. The method according to claim 1, wherein the exogenous agent is a pharmaceutical composition comprising a glucocorticoid, an estrogen, a progestin, a retinoid, or an ecdysone, or an analog or mimetic thereof.

7. The method according to claim 1, wherein the one or more nucleic acid molecules further comprises at least one nuclear localization signal (NLS).

8. The method according to claim 1, wherein the one or more nucleic acid molecules further comprise a tissue-specific promoter directing expression of the activation domain.

9. The method according to claim 1, wherein the one or more nucleic acid molecules are delivered via a non-viral delivery system.

10. The method according to claim 1, wherein the one or more nucleic acid molecules are delivered via a viral delivery system.

11. The method according to claim 10, wherein the viral delivery system is selected from adenovirus, lentivirus, or adeno-associated virus.

12. The method according to claim 11, wherein the viral delivery system comprises at least one recombinant adeno-associated virus stock.

13. The method according to claim 1, wherein the one or more nucleic acid molecules are delivered via a combination of a non-viral and a viral delivery.

14. A gene editing system comprising one or more recombinant adeno-associated viral (rAAV) vector stocks, said system comprising:
(a) at least one nucleic acid sequence encoding one or more DNA binding domains,
(b) at least one nucleic acid sequence comprising a coding sequence of an activation domain for a regulatable promoter;
(c) at least one coding sequence encoding a meganuclease; wherein expression of the meganuclease is under the control of at least one regulatable promoter, wherein the promoter is activated and/or regulated by pharmaceutical agent; and
(d) optionally a nucleic acid sequence comprising a donor gene for insertion into a selected gene locus.

15. The gene editing system according to claim 14, wherein the system comprises at least two AAV stocks, each of which has the same AAV capsid.

16. The gene editing system according to claim 15, wherein the system is designed for targeting to the liver and comprises rAAV having a capsid from a Clade E AAV.

17. The gene editing systems according to claim 14 for use in a method for treating a disease, disorder, or condition in a subject.

18. (canceled)
